(19) United States
(12) Patent Application Publication
(10) Pub. No.: US 2010/0285518 A1
(43) Pub. Date: Nov. 11, 2010

(54) PHOTOACOUSTIC DETECTION OF ANALYTES IN SOLID TISSUE AND DETECTION SYSTEM

(75) Inventors: John A. Viator, Columbia, MO (US); Devin McCormack, Columbia, MO (US); Paul S. Dale, Columbia, MO (US)

Correspondence Address: GREER, BURNS & CRAIN, 300 S WACKER DR, 25TH FLOOR, CHICAGO, IL, 60606, US

(73) Assignee: THE CURATORS OF THE UNIVERSITY OF MISSOURI, Columbia, MO (US)

(21) Appl. No.: 12/763,700

(22) Filed: Apr. 20, 2010

Related U.S. Application Data
(60) Provisional application No. 61/170,880, filed on Apr. 20, 2009.

(51) Int. Cl. C12Q 1/02; C12M 1/34

(52) U.S. Cl. 435/29; 435/288.7

(57) ABSTRACT

A preferred system for detecting an analyte in solid tissue, such as an intact lymph node, in vitro includes a laser arranged to generate a pulsed laser beam into solid tissue, which can be a fully intact lymph node. An acoustic sensor, and preferably at least three acoustic sensors are arranged in different positions to span a three dimensional space, such as in an X, Y and Z coordinate system, to detect photoacoustic signals generated within the lymph node. At least one computer receives signals from the acoustic sensor(s). The computer determines the presence or absence of, and preferably the position of analyte, from the signals and the timing of the signals. A preferred method for detecting an analyte in a lymph node in vitro includes exposing an extracted lymph node to a pulsed laser beam. A photoacoustic signal is sensed. The photoacoustic signal is analyzed to confirm the presence or absence of an analyte in the lymph node. Preferably, multiple photoacoustic signals are sensed from sensors that span a three dimensional space and the position of analyte is also determined.

### PRIORITY CLAIM AND REFERENCE TO RELATED APPLICATION

The application claims priority under 35 U.S.C. §119 from prior provisional application Ser. No. 61/170,880, which was filed Apr. 20, 2009.

### FIELD OF THE INVENTION

Fields of the invention is analyte detection in solid tissue and tissue analysis systems. A preferred application of the invention is the in vitro detection of melanoma micro-metastasis in intact excised sentinel lymph nodes, and a preferred system of the invention is a photoacoustic system that can detect both the presence and geographical location of melanoma micro-metastasis in extracted lymph nodes.

### BACKGROUND

A sentinel lymph node is the node from a first group of nodes that is reached by metastasizing cancer cells that travel from a cancerous tumor through the lymphatic system. Sentinel lymph node mapping involves detecting a sentinel lymph node, typically via dye injection, and removing the sentinel lymph node for a biopsy to determine the presence or absence of metastasizing cancer cells. The idea of lymphatic mapping is based upon the concept that sites of cutaneous melanoma and other cancers have specific patterns of lymphatic spread and that one or more nodes are the first to be involved with metastatic disease within a given lymph node basin. If these first or sentinel lymph nodes are not involved, the entire basin should be free of tumor. This type of procedure is in currently used to diagnosis and treat malignant melanoma and breast cancer, two types of cancer that can be detected with sentinel lymph node mapping. Detection and monitoring of metastatic disease is crucial for positive clinical outcomes in treatment of these and other forms of cancer. Knowledge of the regional lymph node status is important not only for prognosis but also to determine therapy.

Melanoma is the deadliest form of skin cancer and has the fastest growth rate of all cancer types. In the U.S., the lifetime risk of getting melanoma is about 1 in 55, while in other parts of the world it is even greater. Early surgical resection of melanoma is the best avenue of therapy. Detection and monitoring of metastatic disease is crucial for positive clinical outcomes, however.

A weakness in the sentinel node mapping technique is the biopsy used to determine whether an extracted sentinel lymph node has metastasized cancer cells. This is especially true in early stages when a sentinel lymph node may have only a small number of micro-metastasized cells. Such cells make up a very small volume of a lymph node, and are difficult to detect when a lymph node is sectioned during a biopsy. A typical biopsy involves taking eight to ten sections, providing scant opportunity to detect such micro-metastasized cells. High false negative rates can be expected as the lymph node is only examined in part. Once the lymph node is removed, typically six to ten sections of approximately 6 μm thickness are taken and examined for metastasis. Thus, in a typical node with a 1 cm length, only a very small fraction (sometimes less than 1%) of the node is subjected to testing. Even a large increase in the number of sections, impractical in reality, would still rely largely upon chance to detect micro-metastasized cells. Immunohistochemical staining for the melanoma markers further enhances sensitivity, but a fair percentage of biopsies will still provide a false negative even when optimal techniques are used. Studies have found false negatives exceed 10% of sentinel node biopsies. See, Jansen, L., Nieweg, O., Peterse, J., Hoefnagel, C., Olmos, R., and Kroon, B., “Reliability of Sentinel Lymph Node Biopsy for Staging Melanoma,” Br. J. Surg., 87, pp. 484-489 (2000); Yu, L., Flotte, T., Tanabe, K., Gadd, M., Cosimi, A., Sober, A., Mihm, M., Jr., and Duncan, L., “Detection of Microscopic Melanoma Metastases in Sentinel Lymph Nodes,” Cancer, 86, pp. 617-627 (1999).

Efforts have been made to improve the detection of sentinel nodes and metastases. As en example, reverse transcriptase polymerase chain reaction has been used to detect melanoma precursors in sentinel node biopsies, but the utility of RT-PCR in clinical testing is unclear. See, Hauschild, A., and Christophers, E., “Sentinel Node Biopsy in Melanoma,” Virchows Arch., 438, pp. 99-106 (2001). Wang et al. have proposed the use of ultrasound modulated optical tomography to detect sentinel nodes for melanoma and breast cancer, but this technique will not detect of micro-metastasis. Wang, et al., “Sentinel Lymph Node Detection ex vivo Using Ultrasound-Modulated Optical Tomography,” J. Biomed. Opt., 13 (2008). A related in vivo technique locates sentinel lymph nodes. Wang, et al., also proposed a noninvasive technique to locate sentinel lymph nodes in vivo via a noninvasive photoacoustic identification system with methylene blue injection, but this technique identifies the position sentinel lymph nodes in vivo by detecting the methylene blue that collects in the sentinel lymph node after injection. See, Wang et al., “Noninvasive Photoacoustic Identification of Sentinel Lymph Nodes Containing Methylene Blue in vivo in a Rat Model,” Journal of Biomedical Optics 13 (5), 054033 (September/October 2008). Wang et al. identify a sentinel lymph node by injecting methylene blue dye into the tissue where the original tumor was resected. The dye is taken up by the lymphatic vessels that drained the tumor and lead to the sentinel lymph node (SLN). The blue dye is used as a photoacoustic target, and detection of the dye permits use of a fine needle aspiration to take a biopsy without cutting into the axilla. This technique can potentially reduce the invasiveness of evaluating a SLN. Ex vivo tests were conducted to test the sensitivity of dye detection. The technique is not suitable for the location of micro-metastasis in an extracted lymph node as it is the exogenous dye to find the SLN in vivo. The dye non-selectively colors the SLN. Another attempt to use ultrasound to detect metastasis in sentinel lymph nodes demonstrated a false positive rate of 61%. Rossi et al., “The Role of Preoperative Ultrasound Scan in Detecting Lymph Node Metastasis Before Sentinel Node Biopsy in Melanoma Patients,” J. Surg. Oncol., 83, pp. 80-84.

### SUMMARY OF THE INVENTION

A preferred system for detecting an analyte in solid tissue, such as an intact lymph node, in vitro includes a laser arranged to generate a pulsed laser beam into solid tissue, which can be a fully intact lymph node. An acoustic sensor, and preferably at least three acoustic sensors are arranged in different positions to span a three dimensional space, such as in an X, Y and Z coordinate system, to detect photoacoustic signals generated within the lymph node. At least one computer receives signals from the acoustic sensor(s). The computer determines the presence or absence of, and preferably the position of analyte, from the signals and the timing of the signals.

A preferred method for detecting an analyte in a lymph node in vitro includes exposing an extracted lymph node to a pulsed laser beam. A photoacoustic signal is sensed. The photoacoustic signal is analyzed to confirm the presence or absence of an analyte in the lymph node. Preferably, multiple photoacoustic signals are sensed from sensors that span a three dimensional space and the position of analyte is also determined.

### BRIEF DESCRIPTION OF THE DRAWINGS

FIG. 1 is a schematic diagram of a preferred embodiment photoacoustic detection system for detection of analytes in solid tissue;

FIGS. 2A-2C are plots of photoacoustic response taken from a healthy canine lymph node in an experimental three sensor system in accordance with FIG. 1, and FIGS. 2D-2F are plots showing the photoacoustic response after the injection of melanin cells into the lymph nodes; and

FIG. 3 includes plots of signal strengths for pig lymph node testing in an experimental three sensor system in accordance with FIG. 1.

### DETAILED DESCRIPTION OF THE PREFERRED EMBODIMENTS

The invention provides methods and systems for photoacoustic detection of analytes in extracted solid tissues, such as sentinel lymph nodes. Methods and systems of the invention are capable of detecting and geographically locating microscopic analytes in lymph nodes. Any analyte that is a light absorber can be detected in solid tissues, but methods and system of the invention are especially useful to detect the presence and position of micro-metastases in lymph nodes. Methods and systems of the invention can, for example, detect the presence or absence of and geographically locate in three dimensions the position of micro-metastases in extracted sentinel lymph nodes as replacement or aid to a traditional sentinel node biopsy.

A preferred system for detecting an analyte in solid tissue, such as an intact lymph node in vitro, includes a laser arranged to generate a pulsed laser beam into the solid tissue, which can be a fully intact lymph node. An acoustic sensor, and preferably at least three acoustic sensors are arranged in different positions to span a three dimensional space, such as an X, Y and Z coordinate system, to detect photoacoustic signals generated within the lymph node. At least one computer receives signals from the acoustic sensor(s). The computer determines the presence or absence of, and preferably the position of analyte, from the signals and the timing of the signals.

A method for detecting an analyte in a lymph node in vitro includes exposing an extracted lymph node to a pulsed laser beam. A photoacoustic signal is sensed. The photoacoustic signal is analyzed to confirm the presence or absence of an analyte in the lymph node. Preferably, multiple photoacoustic signals are sensed from sensors that span a three dimensional space and the position of analyte is also determined. Preferred methods and systems of the invention place an extracted lymph node in an acoustic medium and then sense photoacoustic response from the acoustic medium. A preferred acoustic medium is de-ionized water. Gels and oils, e.g., mineral oil, can also be used as an acoustic medium. Air is an acoustic medium as well, though liquid, gel and oil mediums are preferred. In other embodiments, a node can be suspended in air (pinned or otherwise supported) and photoacoustic transducers are in physical contact with the node itself, preferably with some acoustic matching gel.

Preferred embodiment systems and methods of the invention detect melanoma micro-metastasis in extracted lymph nodes. The analyte in that case is the micro-metastasis itself. These preferred methods and systems of the invention use melanoma's inherent optical absorption to find metastasis once the SLN is resected. The optical absorption of melanoma cells is utilized to generate the necessary photoacoustic response for detection. This provides a very powerful technique for the detection of melanoma micro-metastasis.

In other preferred embodiments, other types of cancer cells can be detected. Breast cancer cells or other types of cancer cells can be detected in another embodiment. In this instance, an exogenous absorber is introduced. The exogenous absorber is one that is specifically attracted to the cancer cells of interest. Example exogenous absorbers include nanoparticles functionalized to known antigens on the cancer cells (such as HER-2 for some breast cancers, or estrogen receptors in estrogen positive breast cancer cells). These nanoparticles can be gold, silver or other nanoparticles. Functionalized quantum dots or microspheres can also be used. Histochemical dyes that specifically color the targeted cancer cells are another exogenous receptor that can specifically target cancer cells and act as an absorber.

Preferred embodiments of the invention will now be discussed with respect to the drawings. The drawings may include schematic representations, which will be understood by artisans in view of the general knowledge in the art and the description that follows. Features may be exaggerated in the drawings for emphasis, and features may not be to scale. Experimental systems will be discussed, and artisans will recognize broader feature of the invention from the experimental systems and test results.

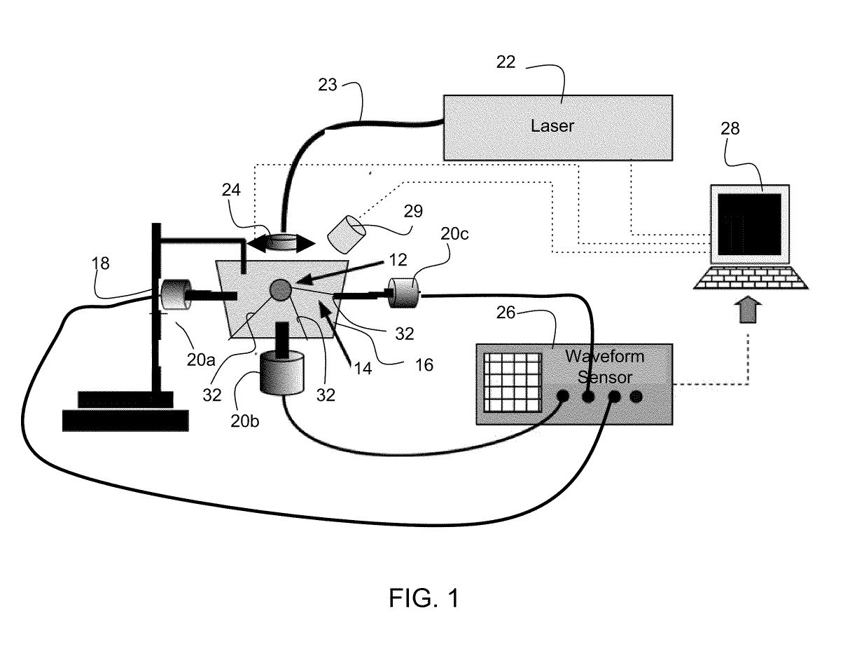

FIG. 1 shows an example system 10 for detecting an analyte in solid tissue, such as an intact lymph node in vitro. Solid tissue in the form of an intact lymph node 12 or a substantial portion of an intact lymph node is positioned a predetermined sample volume location in an acoustic medium 14 contained in a sample holder 16. The sample holder is suspended by a stand 18 that can provide isolation from external mechanical vibrations. Generally, vibrations won't affect measurements, though, because the sensed signals are in the range of tens of megahertz.

Acoustic sensors 20a, 20b, and 20c are arranged at three different positions in an X, Y, Z coordinate system to sense acoustic signals generated in the intact lymph node 12. The preferred system has the three sensors 20a, 20b, 20c to produce independent signals to permit determination of position as well as the presence or absence of analyte, however, the detection of the presence or absence of a photo-absorbing analyte only requires a single sensor. Preferred acoustic sensors are piezoelectric sensors. Sensors other than piezoelectric may be used, with examples including detectors that measure optical perturbations in the sample or carrier fluid surrounding the node.

The photoacoustic signals are induced by a pulsed laser beam produced by a laser 22. The beam of the laser is carried through an optical fiber 23 and can be collimated by a lens 24 to be directed at the lymph node 12. Collimation is preferable but not necessary. Collimation can help to maintain a high laser fluence, which is desirable, but it is not crucial because a sufficiently focused beam is provided from the optical fiber. In one embodiment, the lens 24 creates a beam that encompasses the entire volume of the lymph node 12. In other embodiments, the lens 24 creates a narrowly focused beam, which is then scanned in a pattern to “image” the entire lymph node 12. Scanning a narrowly focused beam will provide increased propagation of photons through the lymph node 12 and any micro-metastases present in the lymph node 12, but either embodiment can effectively locate in three dimensions, such as in X, Y and Z space, the position of any micro-metastases in the lymph node 12. Scanning permits laser light to enter the turbid medium of the lymph node closer to the micro-metastasis, increasing the photoacoustic response. A scanning micromotor 25 can create the relative movement between the sample node 12 and the laser beam by moving the collimation lens 24, which can be controlled by the computer 28. The fiber-lens scanned to irradiate the entire node 12 in a scan pattern, though a scan can be performed using other methods, such as by translating the node or steering the laser beam. A photosensor associated with the laser 22 can also be used to trigger the computer 28 and waveform sensor 26. Any micro-metastases are revealed by distinct acoustic waves sensed by a waveform analyzer 26 and analyzed by a computer 28. With the three sensors 20a, 20b, and 20c at unique locations that span a three dimensional space, e.g., in an X, Y, and Z coordinate system, the computer 28 can determine the location of any micro-metastases within the lymph node 12 by the timing of the acoustic wave received by each of the sensors 20a, 20b, and 20c. The three sensors 20a, 20b, and 20c should be orthogonal, though as long as they are not collinear, backprojection can be used to determine the position of analyte. Specifically, as long as the three vectors determined by the sensors' 20a, 20b, 20c direction span a three dimensional space, there will be sufficient information to conduct backprojection calculations. The distances between the sensors and the node are, for the most part, unimportant, however larger distances product a larger device and there can also be undesirable viscoelastic attenuation in the coupling fluid. Generally, it is preferred that there be about a centimeter or less distance between the sensors 20a, 20b, and 20c and the node 12.

The computer 28 has knowledge of the location of the three sensors 20a, 20b, and 20c and the speed of travel of the signal within the node and acoustic medium 14 surrounding the node, which permits determination of a location of the melanoma within the node can be made. The computer 28 can perform an automatic scan and output or store positional information concerning analyte. The output or stored information can take the form of a map, for example, which maps the node in three dimensions and provides an indication of the position of any analyte. The photoacoustic information can be used to provide a map of the analyte position, e.g., a map of detected melanoma. This information can be overlaid onto a node image obtained, for example by standard imaging by the camera or other optical sensor 29, to show where the melanoma is within the node.

More than three sensors can be used, with other examples including 5, 7, 10, or even dozens. It is useful to know the location of the sensors relative to the node and to know the relative time that the signal is detected by each sensor. All sensors can be arranged to be on a common time scale so that the relative difference between reception of the signal at a first and second sensor can be determined. Generally, a larger number of sensors offers a greater degree of accuracy in estimating the location of the melanoma. Greater number of sensors, however, also can lead to greater complexity and cost. Also, the degree of accuracy of estimating the location of the melanoma in many applications may not be so great as to require more than 3, 4, 5, 6, or 7 sensors.

The sample holder 16 can be a test chamber that is configured to contain liquid acoustic medium 14 and can have a variety of geometries. The acoustic medium 14 should be transparent to the laser wavelength that is used (so that the acoustic medium does not act as an absorber. Saline solution is an example suitable medium for many laser wavelengths. Preferably, the acoustic medium has an acoustic impedance that is substantially matched to that of the tissue being examined. The sample holder may be transparent, or can have a transparent section for accepting the laser beam. The lymph node should be positioned within the sample holder at a known location, so that the results of the sensor detection can be correlated to a location in the node. In preferred embodiments, pins 32 or other elements that are transparent to the laser wavelength being used can be used to pierce or otherwise hold a node. In addition to holding the node in a particular predetermined positions, the pins can also provide a three dimensional location reference. The sample holder 16 preferably includes other landmarks so that a relative position of the node to the test chamber is known. Coordinate system markings in the sample holder can be useful to establish known locations in three dimensions (e.g., an X, Y and Z axis). These can be useful to coordinate the estimated location of the detected analyte in the node to a known position for later detailed sectioning of the node. The location of the sensors 20a, 20b, 20c relative to the sample holder coordinate system is provided to the computer 28. The system preferably includes a camera or other form of optical sensor 29 that images the node 12 and the landmarks. Using this image, the computer can generate a three dimensional map of the node that can be combined with a map of analyte positions generated from the photoacoustic signals. In other embodiments, the system can use a different wavelength from the laser 22 to photoacoustically image the node 12. The wavelength used could target water content in the node for absorption, for example. In this case, the acoustic medium should not be water, and a suitable gel or oil could be used.

Example markings can serve as landmarks to assist node position include may include grid or other reference markings or marker elements arranged along first and second planes that the node can be positioned relative to and that can be useful to establish a three dimensional X, Y and Z coordinate system and positions. Other example marker elements include pins, posts or other structural elements rising vertically from a sample holder floor, vertical walls or ridges with markings, posts or pins extending horizontally into a sample holder from a sidewall, or other physical marker elements that the node may be positioned relative to in three dimensions within a sample holder. Nylon pins are one example, since they do not absorb laser light. Other holding elements made of non-light absorbing materials, with some polymers being examples, may be used. Each pin 32 can either determine a coordinate or the pins 32 can be used in such a way that the sensors 20a, 20b, 20c form the X, Y, and Z coordinates.

The sensors 20a, 20b, 20c may be arranged along a sample holder sidewall, floor, top wall, or otherwise in fluid contact with the carrier fluid to detect pressure waves in the fluid. Or, other sensors that detect photoacoustic events through deflection of a light beam may be used which do not require fluid contact. These may be arranged outside of the test sample holder.

Some systems may include a second station for detailed sectioning of the node in the estimated location of the melanoma. This represents a significant advantage over the prior art, in that highly detailed sectioning of the node can be directed to only the particular location of the melanoma in the node. Significant labor and cost savings are achieved.

An experimental system in accordance with FIG. 1 was constructed and tested. The discussion of the experiments will reveal additional features of preferred systems and methods, while artisans will appreciate that commercial systems in accordance with the invention can be constructed with specially fabricated components to the advantage of performance, compactness and conventional optimizations. While the experiments and preferred embodiments are directed toward the detection of melanoma, artisans will appreciate that other nodes and other analytes that are photoelectric energy absorbers can be analyzed with methods and systems of the invention.

### Experimental System and Data

In the experiments, a photoacoustic responses from a lymph nodes with as few as 500 melanoma cells were unambiguously detected and information was obtained from multiple sensors to permit the determination of the location of the cells in the lymph node in the three dimension space of the lymph node. Normal lymph nodes showed no response. Thus, the detection method and system of the invention can be used to detect the presence of micro-metastases in fully intact lymph nodes. It can also be used to guide further histologic study of the node, increasing the accuracy of a sentinel lymph node biopsy. The study showed no false positive or false negative results.

Most melanomas are highly melanotic, with estimates of amelanotic melanoma being less than 5% or 1.8-8.1%, though this latter figure includes partially pigmented melanoma. Thus, the great majority of melanomas contain native light absorbers that can be exploited using photoacoustic generation and detection. A photoacoustic effect occurs when the optical energy of a photon is transduced into a mechanical disturbance, resulting in an acoustic wave.

Experimental Detection System

A frequency-tripled Q-switched Nd:YAG laser (Vibrant 355 II, Opotek, Carlsbad, Calif.) was used to pump an optical parametric oscillator. This system had a wavelength range of 410-2400 nm. For these experiments, the system was set at a wavelength of 532 nm and was focused through a 600 μm diameter fiber to irradiate lymph nodes as in FIG. 1. The laser energy ranged from 4-6 mJ and the laser pulse duration was 5 ns. The laser system had a repetition rate of 10 Hz. The photoacoustic signals generated in the lymph nodes were received by three piezoelectric acoustic sensors made from polyvinylidene fluoride (PVDF) film (Ktech Corp., Albuquerque, N. Mex.). The signals were transmitted to an oscilloscope (TDS 2024, Tektronix, Wilsonville, Oreg.) triggered by photodiode (DET10A, Thorlabs, Newton, N.J.) monitoring the laser output. The fiber was positioned above the lymph node at approximately 1 cm. The acoustic sensors were placed orthogonally about the lymph node, each sensor at a distance between 1-3 mm from the closest lymph node surface. A more precise position can be deduced from each waveform by the product of the time of the photoacoustic wave and the speed of sound in tissue, which is approximately 1.5 mm/ns. The transducers were made with segments of semirigid coaxial cable (Micro-coax, Pottstown, Pa.) approximately 10 cm long. The procedure for making acoustic sensors from PVDF and coaxial cable is described more fully in J. Viator, et al, “Clinical Testing of a Photoacoustic Probe for Port Wine Stain Depth Determination,” Lasers Surg. Med., 30, pp. 141-148 (2002). In the experiments, the outer conductor diameter was 3.6 mm and the inner conductor diameter was 0.9 mm with the two conductors being separated by a dielectric. A 25 μm thick PVDF film was attached to the exposed polished face of the coaxial cable.

Lymph Node Preparation and Testing

A canine lymph node was separated from connective and other tissues surrounding the node, and then soaked overnight in de-ionized water to remove any blood in or around the lymph node. The lymph node was about 1 cm long in the shape of a lima bean. The entire lymph node was placed in an acoustic medium that was a deionized water bath, which ensured acoustic propagation to the sensors. A wavelength of 532 nm was directed through a 600 μm diameter fiber and at the top surface of the lymph node. The sensed signal was amplified five times using a 350 MHz instrumentation amplifier (SR445, Stanford Research Systems, Sunnyvale, Calif.) and then was averaged 128 times. With a 10 Hz laser repetition rate, one acquisition with 128 averages took 12.8 s.

Canine Lymph Node with Large Melanoma Pellet

To test the ability of the system to detect melanoma, the lymph node was injected with melanoma cells. A culture of malignant human melanoma cell line HS 936 served as a source of the melanoma cells. A high concentration melanoma suspension was spinned down by centrifuge until the melanoma formed a pellet. The excess solution was removed and a high concentration of melanoma was drawn out by pipet. The total number of melanoma cells was approximately 1×10⁶. This cellular mass was approximately 1 mm in diameter. The laser spot on the lymph node was approximately 1.5 mm in diameter. The laser beam was scanned to irradiate the entire node. The 1.5 mm spot diffused to a larger area within the nodes, but scanning of the laser beam was used to irradiate the entire node. A small incision was made on the lateral surface of the lymph node, and the melanoma was injected into the incision by pipet to simulate a micro-metastasis. The tests, including making incisions, were repeated on a control lymph node in which no melanoma was implanted.

Pig Lymph Nodes with Small Melanoma Pellets

Spheres were also formed from much smaller numbers of melanoma cells. This procedure was different than the centrifuge discussed above. Specifically, melanoma cells were collected in a suspension in an acrylamide solution. This acrylamide solution was solidified into spheres of approximately 1 mm diameter using ammonium persulfate and Tetramethylethylenediamine (TEMED), both from Sigma Aldrich, St. Louis, Mo. The uninitiated acrylamide solution with suspended cells was dropped into mineral oil, creating spheres that solidified within 1 min due to the ammonium persulfate and TEMED. Each sphere formed from this technique contained approximately 500 melanoma cells and was implanted within healthy pig lymph nodes. The lymph nodes from healthy from healthy pigs had similar size and shape to the canine lymph node. Each “positive” lymph node was implanted with melanoma cells.

A higher amplification of the sensor signals was used for these measurements (×125). The optical fiber was positioned approximately 1 mm above the lymph nodes, making a spot of about 600 μm in diameter. Thus the laser fluence at the lymph node surface for the pig lymph nodes was approximately six times higher than it was for the canine lymph node.

Results and Discussion

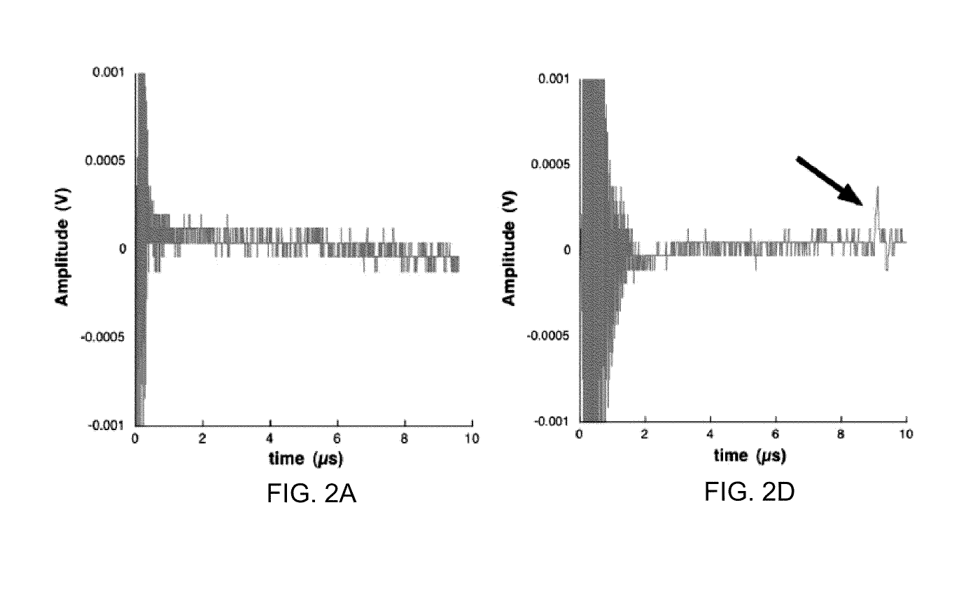

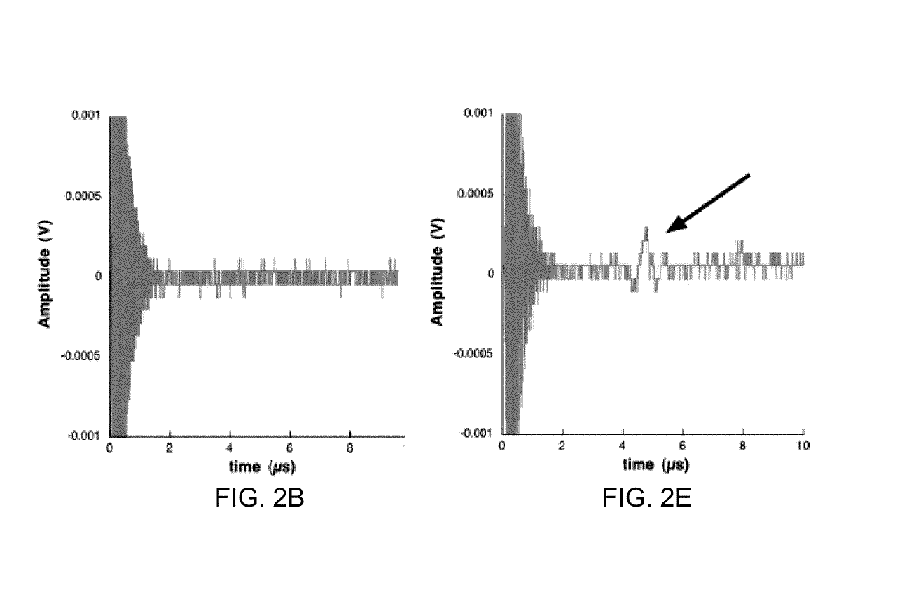

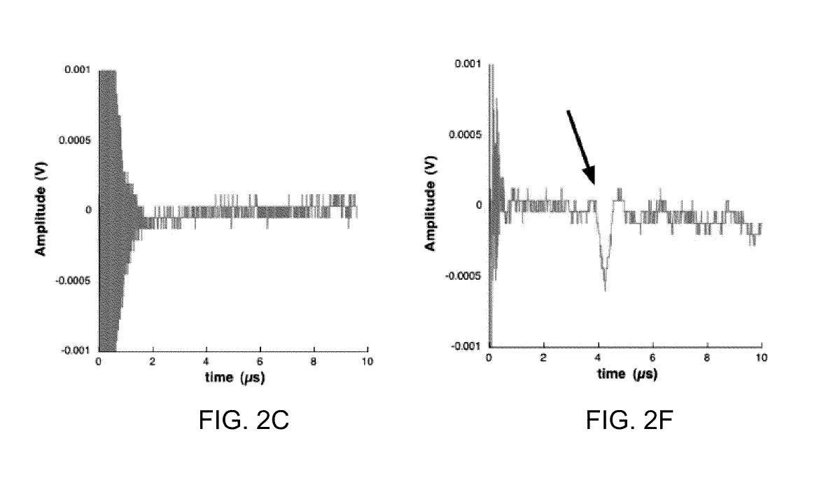

FIGS. 2A-2C show photoacoustic waveforms from the three respective sensors obtained after irradiating the canine lymph node but prior to injection of melanoma cells. FIGS. 2D-2F show wave forms from the respective sensors after injection of melanoma cells. The initial waveform that occurs within 1 μs is due to electrical noise from the laser. For the lymph node with no melanoma added, as seen in FIGS. 2A-2C, there are no photoacoustic signals. The signals show only a flat line with a baseline noise level of approximately 100 μV. As seen in FIGS. 2D-2F, for the lymph node with the melanoma cells present, there are three appreciable photoacoustic signals. For detector 1, as seen in FIG. 2D, the signal occurs at about 9 ns with a peak to peak amplitude of about 0.5 mV. For detector 2, as seen in FIG. 2E, the signal occurs at about 4.5 μs with an amplitude of about 0.4 mV. For detector 3, as seen in FIG. 2F, the signal occurs at about 4.2 μs with an amplitude of about 0.6 mV. The waveform from detector 3 is inverted due to acoustic diffraction. However, it is only the presence of the wave that is needed for detection and the timing of the wave that is need for positional determination, thus the wave shape is irrelevant.

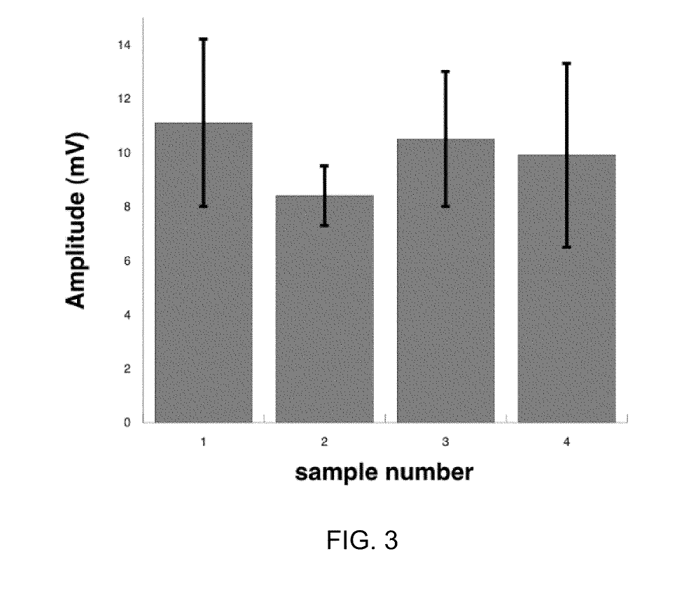

The signal strengths from the pig lymph nodes are shown in FIG. 3. Each lymph node signal comprises an average of eight measurements. The control lymph nodes, in which no melanoma was implanted showed so signals, similar to the control waveforms shown in FIG. 3. The results from the pig lymph nodes clearly showed that small numbers of melanoma cells create photoacoustic signals when irradiated with nanosecond duration laser light. In the pig lymph nodes, there were approximately 500 melanoma cells. With an average diameter of about 20 nm, such a micro-metastasis would be about 100-200 μm in diameter. This number of cells constitutes a small mass that is found only by microscopic inspection of stained sections. Such a micro-metastasis can easily be missed in histological sectioning of a 1 cm long node. The strong and clear signals indicate that even smaller number of cells should be detectable.

### Photoacoustic Backprojection for Precise Localization

One technique for to determine a specific location of a micro-metastases within a lymph node is photoacoustic backprojection, which can therefore be used to guide a histological examination and decrease false negative screens. Using backprojection reconstruction, it is possible to localize the metastasis and determine its location within the node so that histological sections can be chosen for the highest probability of detection for histological examination. Backprojection is a mathematical process that is similar to triangulating a signal using different locations. In addition, filtering and denoising can be performed. A suitable backprojection technique is disclosed in “Iterative Reconstruction Algorithm for Optoacoustic Imaging,” J. Acoust. Soc. Am. Volume 112, Issue 4, pp. 1536-1544 (October 2002).

### Sensitivity Optimization

The experiments showed no false positive rate. However, in practice, incomplete rinsing of the nodes could result in residual blood being present in the nodes. To avoid the blood contributing an unwanted photoacoustic response, the laser wavelength can be change to red, e.g., 630 nm, to reduce the photoacoustic response from deoxygenated hemoglobin by a factor of about eight and the oxygenated response by a factor of more than 50. The melanin response, however, would only reduce by about a factor of two. Thus, sensitivity is increased while noise is limited.

Another option is to use two wavelengths and analyze the relative response of the two wavelengths. For example, responses to 532 nm and 630 nm wavelengths could be taken to classify photoacoustic waves as arising from hemoglobin or melanin. A statistical classification has been used to discriminate thermally coagulated blood and viable hemoglobin. See, Viator, et al. “Photoacoustic Discrimination of Viable and Thermally Coagulated Blood Using a Two-Wavelength Method for Burn Injury Monitoring,” Phys. Med. Biol., 52, pp. 1815-1829 (2007). Use of two wavelengths can similarly be used to distinguish between blood and melanin. The unique absorption spectrum of hemoglobin in contrast to the simple spectrum of melanin makes such a classification possible.

The experiments described above showed that the set up for the pig nodes compared to the canine node increased detection. The difference in the two setups was increased amplification from 5 times to 125 times and by increasing the laser fluence by closer placement of the optical fiber to the tissue surface. Furthermore, the sensors built for the pig nodes were several times more sensitive to acoustic waves. The pig sensors had the same basic construction of the canine sensors, but were constructed for higher sensitivity. These improvements gave us an increase in sensitivity of about three orders of magnitude.

Wavelet denoising can be used to increase the signal to noise ratio as well as scanning during detection. Suitable denoising and scanning is disclosed in Viator et al., “Automated Wavelet Denoising of Photoacoustic Signals for Circulating Melanoma Cell Detection and Burn Image Reconstruction”, Phys. Med. Biol., pp. N227-N236 (May 21, 2008).

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A method for detecting an analyte in solid tissue in vitro comprising:
placing the solid tissue at a predetermined position;
irradiating the solid tissue with a pulsed wavelength of light selected to pass through the solid tissue with insubstantial absorption and to be at least partially absorbed by the analyte;
acoustically sensing a photoacoustic response generated by said step of irradiating for a period of time to obtain a photoacoustic waveform;
analyzing the photoacoustic acoustic waveform and attributing a detected response peak to the presence of analyte.

2. The method of claim 1, wherein said step of placing comprises placing the solid tissue at a predetermined position in an acoustic medium and said step of acoustically sensing comprises sensing the photoacoustic response through the acoustic medium.

3. The method of claim 2, wherein the acoustic medium comprises de-ionized water.

4. The method of claim 2, wherein the acoustic medium comprises an oil or gel.

5. The method of claim 1, wherein said step of analyzing comprises determining a location of any detected analyte based upon the timing of a detected response peak.

6. The method of claim 1 wherein said step of acoustically sensing comprises obtaining a plurality of unique photoacoustic responses and said step of analyzing determines the presence or absence of analyte in addition to the determining the location of any detected analyte.

7. The method of claim 1, wherein said step of irradiating comprises scanning a laser beam over the solid tissue.

8. The method of claim 1, wherein said step of irradiating comprises focusing a laser beam upon the solid tissue.

9. The method of claim 5, wherein the laser beam comprises a pulsed laser beam emitting in the red wavelength range.

10. The method of claim 5, wherein said focusing comprises emitting the laser beam through an optical fiber within one to a few millimeters of the solid tissue.

11. The method of claim 1, wherein the solid tissue comprises an intact lymph node or a substantial portion of an intact lymph node.

12. The method of claim 8, wherein the analyte comprises melanin.

13. The method of claim 1, wherein said step of irradiating comprises irradiating the solid tissue with multiple pulsed wavelengths of light.

14. A system for detecting an analyte in solid tissue in vitro comprising:
a sample holder configured to hold a solid tissue at a predetermined volume in the sample holder;
a laser that can generate a pulsed laser beam;
an optical focuser for directing the pulsed laser beam into the solid tissue;
an acoustic sensor positioned for detecting photoacoustic response of analyte contained in solid tissue; and
a computer for analyzing the photoacoustic response and determining the presence or absence of analyte in the solid tissue based upon the photoacoustic response.

15. The system of claim 12, wherein said sample holder comprises one or more pins for holding solid tissue at the predetermined volume and said one or more pins are substantially transparent to the pulsed laser beam.

16. The system of claim 15, wherein said sample holder is configured to contain an acoustic medium and hold the solid tissue at the predetermined volume in the acoustic medium and said acoustic sensor is positioned to be in acoustic contact with an acoustic medium held by the sample holder.

17. The system of claim 14, wherein said acoustic sensor comprises at least three acoustic sensors that are not collinear and said computer receives independent signals from the at least three acoustic sensors and uses the independent signals to determine the presence or absence and position of analyte from the signals and the timing of the signals.

18. The system of claim 14, wherein said acoustic sensor comprises at least three acoustic sensors arranged to span a three-dimensional space and said computer receives independent signals from the at least three acoustic sensors and uses the independent signals to determine the presence or absence and position of analyte from the signals and the timing of the signals.

19. The system of claim 18, wherein said at least three acoustic sensors are arranged in different positions along an X, Y and Z coordinate system.

20. The system of claim 14, wherein the sample holder comprises marker elements that define a three dimensional coordinate system, the solid tissue positioned at a known position in the three dimensional coordinate system, the at least three sensors are arranged in known positions relative to the defined three dimensional coordinate system.

21. The system of claim 20, further comprising a camera or optical sensor for imaging the solid issue, wherein the computer using the imaging of the solid tissue and the photoacoustic response to generate a map of the solid tissue and the position of analyte within the solid tissue.

22. The system of claim 14, wherein the optical focuser comprises an optical fiber that emits the pulsed laser beam within one or a few millimeters of the predetermined volume.

23. The system of claim 22, further comprising a scanning motor for scanning the pulsed laser beam with respect to the predetermined volume.

24. The system of claim 23, wherein the acoustic sensor comprises coaxial conductors separated by a dielectric and terminating in am exposed polished face with a thin acoustically sensitive film covering the polished face.

25. The system of claim 24, wherein said acoustically sensitive film comprises polyvinylidene fluoride.

26. A method for detecting an analyte in solid tissue in vitro comprising:
exposing extracted solid tissue to a pulsed laser beam;
detecting a photoacoustic signal resulting from said step of exposing; and,
analyzing the photoacoustic signal to confirm the presence or absence of an analyte in the solid tissue.

27. The method of claim 26, further comprising analyzing the photoacoustic signal to estimate the location of the analyte within the solid tissue.

28. The method of claim 26, wherein said detecting comprising detecting with a plurality of acoustic sensors placed in different non-collinear locations, and using the time of detection from each sensor and the relative location of each sensor to estimate a location of the analyte in the solid tissue.

29. The method of claim 26, wherein the number of acoustic sensors is at least 3 and the at least three acoustic sensors are located in different orthogonal positions about the solid tissue.

30. The method of claim 26, wherein the solid tissue is a fully intact lymph node.

31. The method of claim 30, further comprising a preliminary step of extracting a lymph node from a subject.
